(19) United States
(12) Patent Application Publication
(10) Pub. No.: US 2012/0121655 A2
(43) Pub. Date: May 17, 2012

(54) Chewable Formulation Comprising Alginate, Bicarbonate And Carbonate

(75) Inventors: Ian Jolliffe, Hull (GB); Suzanne Demski, Reading, Berkshire (GB); David Harvey, Hull (GB); Leanne Dodds, Reading, Berkshire (GB); Sarah Marshall, Reading, Berkshire (GB); Holly Parker, Essex (GB)

(73) Assignee: Reckitt Benckiser Healthcare (UK) Limited, Slough, Berkshire (GB)

(21) Appl. No.: 13/056,248

(22) PCT Filed: Jul. 15, 2009
(86) PCT No.: PCT/GB2009/001746

§ 371 Date: Jul. 1, 2011

(30) Foreign Application Priority Data
Aug. 6, 2008 (UK) 0814376.0

(51) Int. Cl. A61K 9/00; A61P 1/04; A61K 33/00

(52) U.S. Cl. 424/400; 424/715; 424/717

(57) ABSTRACT

The present invention relates to a solid, ingestible composition comprising: a) an alginate; b) a bicarbonate; and c) a carbonate wherein the composition is in the form of a chewable core, a process for preparing said composition, and its use in the treatment of reflux oesophagitis, gastritis, dyspepsia or peptic ulceration or for use as a sustained releasing or targeted delivery composition.

### CROSS-REFERENCE TO RELATED APPLICATIONS

This application is a US National Stage of International Application No. PCT/GB2009/001746, filed 15 Jul. 2009, which claims the benefit of GB 0814376.0, filed 6 Aug. 2008.

### FIELD OF THE INVENTION

The present invention relates to a novel composition comprising an alginate or alginic acid which is suitable for the treatment of reflux oesophagitis, gastritis, dyspepsia or peptic ulceration.

### BACKGROUND OF THE INVENTION

Reflux oesophagitis occurs when small amounts of gastric juice, food and/or bile acids pass into the lower part of the oesophagus and cause oesophageal inflammation accompanied by pain which may manifest itself in the form of heartburn.

One approach to the problem of reflux oesophagitis has been to administer a preparation which on contact with gastric acid generates a carbonated gelatinous foam or raft which floats on the stomach contents. When reflux occurs it is this raft which precedes the stomach contents into the oesophagus, thus protecting the mucosa from further irritation. Known preparations of this type include liquid preparations comprising sodium alginate, sodium or potassium bicarbonate and calcium carbonate. Such compositions are sold under the trade marks GAVISCON and GAVISCON ADVANCE and are described in GB-A-1,524,740 and WO 95/11668.

However, the liquid products can be difficult to consume, and are less convenient requiring an individual to carry with them a bulky glass bottle. The tablets which are available must be stored/sold in blister packs, or solid plastic packs, which can be awkward to use, to prevent moisture absorption.

In addition, tablets containing alginates combined with antacid ingredients have a chalky mouthfeel with some tooth sticking which is disliked by consumers.

Using these products is often embarrassing to sufferers as the medical appearance highlights that they have a minor illness.

### BRIEF SUMMARY OF THE INVENTION

It would, therefore, be desirable to produce a formulation which is not sensitive to moisture, can be provided in convenient packs, has more acceptable mouthfeel characteristics and can be taken discreetly by consumers.

### DETAILED DESCRIPTION OF PREFERRED EMBODIMENTS

According to the present invention there is provided a solid, ingestible composition comprising:

a) an alginate;

b) a bicarbonate; and

c) a carbonate

wherein the composition is in the form of a chewable core.

The composition can further include a polysaccharide material. Typically, the polysaccharide material is selected from the group consisting of xanthan gum, carrageenans, gum arabic, and cellulose derivatives such as sodium carboxymethyl cellulose, pectins, tragacanth, guar gum.

A preferred polysaccharide material is xanthan gum.

The composition can be provided with a coating, which is typically in the form of a hard or soft coating or shell. The coating may provide a smooth surface resulting from one or more coating layers. The coating can be either sugar-containing or sugar-free, and may be provided with one or more flavourings. The coating is typically non-sticky, and preferably remains intact during processing.

Any alginate may be used in the composition of the present invention, but it is especially desirable to use an alkaline metal salt, an alginate, such as sodium or potassium alginate. Preferably a low viscosity grade alginate is used. These are generally agreed alginate of a viscosity of a 10% w/volume aqueous solution, when determined on a group fuelled RVT viscometer using spindle number 3 at 20 rpm at 20° C. falls in the range of 200 to 1500 MPA. An example of a suitable commercial grade of low viscosity sodium alginate is Protanal LFR 5/60, obtainable from FMC Biopolymer. High viscosity grades of alginate may also be used. These are generally grades of alginate for which the viscosity of a 1% weight/volume aqueous solution, when determined on a Brookfield viscometer model RVT using spindle number 3 at 20 rpm at 20° C., with above 5 mp. An example of a suitable commercial grade of high viscosity sodium alginate is Protanal SF 200, also obtainable from FMC.

The composition of the present invention generally has a content of alginate of from 2 to 90 wt %, preferably 5 to 30 wt %, preferably 5 to 15 wt % based on the total weight of the composition.

The composition of the present invention comprises a bicarbonate and a carbonate. Examples of bicarbonates are alkali metal bicarbonates such as sodium and potassium bicarbonate and alkaline earth metal bicarbonates. One or two or more different bicarbonates may be used. Examples of carbonates are alkali metal carbonates such as sodium and potassium carbonate and alkaline earth metal carbonates such as calcium and magnesium carbonate. Further examples are aluminium carbonate and mixed alkali metal carbonates such as sodium glycine carbonate. One or two or more different carbonates may be used. Furthermore one or more bicarbonates may be used with one or more carbonates. Especially preferred combinations are sodium and/or potassium bicarbonate and calcium carbonate.

The carbonate and/or bicarbonate are present in amounts such that they provide an adequate volume of gas (carbon dioxide) to float the gel produced when the alginate contacts the gastric acid in the stomach. The rigidity and thickness of the carbonated alginate raft will depend, for example, upon the relative amounts of carbonate and/or bicarbonate and on the grade of the alginate.

The bicarbonate is generally present in the compositions of the present invention in an amount of from 1.5 to 35 wt %, preferably 2 to 20 wt %, most preferably 3 to 10 wt %. The carbonate is generally present in the compositions of the present invention in an amount of from 0.2 to 55 wt %, preferably 0.5 to 20 wt %, most preferably 1 to 10 wt %.

Preferably the bicarbonate and carbonate may also be present together in the composition, preferably from 1 to 20 wt %, for example in a total amount of from 1 to 40 wt %, preferably 1 to 12 wt %. Approximately equal amounts of the bicarbonate and carbonate may be present in the composition. Alternatively, the composition may comprise more bicarbonate than carbonate. The weight ratio of bicarbonate to carbonate in the composition may be from 1:1 to 2:1.

The compositions of the present invention may also comprise further, optional components.

For example, the compositions of the present invention comprise an insoluble source of divalent and/or trivalent metal ions. Such ions strengthen the raft formed in the stomach. Suitable metal ions are calcium and aluminium. The ions may be provided as part of the bicarbonate and/or carbonate, but may also comprise other anions if desired. For example, suitable sources of calcium ions are calcium carbonate, and suitable sources of aluminium ions are aluminium carbonate, aluminium magnesium carbonate, hydroxide or magaldrate, aluminium sodium carbonate hydroxide or aluminium sodium silicate. If used, the calcium ions are preferably present in an amount of from 8 to 800 parts, and the aluminium ions are preferably present in an amount of from 2 to 500 parts, per 500 parts by weight of alginate.

The compositions of the present invention may also comprise one or more preservatives to prevent contamination and subsequent deterioration by micro-organisms. Examples of suitable preservatives are methyl, ethyl, propyl and butyl para-hydroxybenzoates and their salts, which are preferably used in combinations, for example methyl and propyl or ethyl and butyl. The compositions of the present invention do not need to include such preservative(s), but if a preservative(s) is present it may be used in an amount of, for example, up to 0.5 wt %, based on the total weight of the composition.

The compositions of the present invention may also comprise one or more colourings, sweetenings, flavourings, pH adjusting ingredients and fillers. When the compositions of the present invention are intended for use as sustained releasing compositions they will also comprise at least one active ingredient suitable for specific delivery to the stomach, such as a drug. Examples of suitable drugs are analgesics (e.g. acetaminophen, ibuprofen, flurbiprofen naproxen, diclofenac, ketoprofen, choline salicylate, benzydamine, buprenorphine, hydrocortisone, betamethasone); decongestants (e.g. pseudoephedrine, phenylephrine, oxymetazoline, xylometazoline); cough suppressants (e.g. dextromethorphan, codeine, pholocodine); expectorants (e.g. guaiphenesin, n-acetylcysteine, bromhexine); antiseptics (e.g. triclosan, chloroxylenol, amylmetacresol, hexylresorcinol, dichlorobenzyl alcohol, benzyl alcohol); cardiovascular agents (e.g. glyceryl trinitrate); local anaesthetics (e.g. benzocaine, lignocaine); antacid agents (e.g. calcium carbonate, sodium bicarbonate, magnesium trisilicate, aluminium hydroxide, magaldrate,); antiulcer agents (e.g. carbenoxolone, sucralfate, cimetidine, ranitidine, nizatidine, famotidine, omeprazole, pantoprazole); antihistamines (e.g. loratidine, terfenadine, diphenhydramine, chlorphenhydramine, triprolidine, acrivastine); antinausea agents (e.g. prochlorperazine, sumatriptan); bowel regulatory agents (e.g. diphenoxylate, loperamide, sennosides); antifungal agents (e.g. clotrimazole); antimicrobial agents and antibiotics (e.g. amyl metacresol, DCBA, fusafungine, tyrothricin).

The composition of the present invention may be used in a method of treatment of the human or animal body by therapy, especially in the treatment of reflux oesophagitis, gastritis, dyspepsia or peptic ulceration or for use as a sustained releasing or targeted delivery composition.

The composition of the present invention may be used in the manufacture of a medicament for the treatment of reflux oesophagitis, gastritis, dyspepsia or peptic ulceration or for use as a sustained releasing or targeted delivery composition.

The composition of the present invention may be used in a method of treating reflux oesophagitis, gastritis, dyspepsia or peptic ulceration or for sustained releasing or targeting a delivery composition, which comprises orally administering to a subject in need thereof or liable to need an effective amount of the composition.

The composition is generally administered in an amount of from 100 to 2000 mg alginate per dose.

In accordance with the second aspect of a present invention there is provided in the process for preparing the composition of the first aspect which comprises the steps of:
providing a mixture of excipients suitable for a chewable texture;
heating the resulting mixture to a temperature of not greater than 50° C.;
adding an alginate or alginic acid, and blending the resulting mixture;
adding the bicarbonate and the carbonate, and blending the resulting mixture;
forming the resulting mixture into the desired core product; and
coating the core with a suitable coating material.

Typically the temperature is selected to be not greater than 45° C.

Typically the excipients include water, the content of water being selected such that the composition has the desired consistency.

Preferably xanthan gum is included in the mixture.

The process has a maximum temperature of 50° C. to ensure the stability of active ingredients, particularly the sodium bicarbonate. In most chew forming procedure the process involves forming a sugar solution that whose water content is reduced by strong heating, usually above the boiling point of water. In the process of the present application water is added to give the correct final moisture content to give appropriate final chewing consistency and product stability.

The process can include a first coating step wherein the core is pre-coated with a material such as a gum. The gum is typically selected from the group consisting of xanthan gum, arabic gum, povidone, carrageenans, & cellulose derivatives such as sodium carboxymethyl cellulose, pectins, tragacanth, guar gum.

A hard candy coating can be achieved with sugar or sugar alcohol ingredients in traditional pan coating technologies. In a preferred embodiment, xylitol is used as the coating. Such a coating remains stable without cracking for at least 3 months.

An embodiment of the invention will now be described, by way of example only.

### Example

| Ingredient | Percentage w/w | g/500 g |
|---|---|---|
| Lycasin 80/55 | 20.68 | 103.4 |
| Sorbitan S60P | 0.52 | 2.58 |
| Fat: Softisan 100 | 5.17 | 25.85 |
| Xantural 180 | 1.15 | 5.75 |
| Sodium Alginate | 8.30 | 41.5 |
| Sodium Bicarbonate | 4.45 | 22.25 |
| Calcium Carbonate | 2.67 | 13.35 |
| Mannitol | 33.65 | 168.24 |
| Xylitol | 23.19 | 115.96 |
| Natural mint oil | 0.20 | 1.00 |
| Sucralose | 0.025 | 0.13 |
| Batch Total (g) | 100.00 | 500.00 |

The above formula can be prepared as follows:

The sorbitan is subjected to microwave treatment for twenty seconds. Thereafter, lycasin is added and the mixture is warmed to approximately 50° C. The resulting mixture is now blended. Xantural is added, followed by sodium alginate. The resulting composition is mixed. Mannitol, xylitol, sodium bicarbonate, calcium carbonate, natural mint oil (flavouring) and sucralose (sweetener) are mixed together and half this mixture is added to the alginate—containing mixture and the resultant composition is blended. The remaining mannitol mixture is added and blending for further time. The resulting composition is rolled, cut and shaped. The formulation is then coating using a panning method. The coating can be made of sucrose, isomalt or xylitol.

Optionally, a pre coating step can be included wherein the core formulation is coated with arabic gum. This provides an even surface for adhesion of the outer shell-like coatings, and also acts as a barrier to prevent the core absorbing moisture from the outer coating during the panning stage.

Typically, individual doses of the formulation comprise 3 g of the core material delivering 250 mg sodium alginate.

A polysaccharide has been found to be particularly advantageous in achieving a product with acceptable mould releasing properties from the chewable core forming die units. Of the polysaccharides, xanthan gum was found to be particularly advantageous.

Further modifications and improvements can be incorporated without departing from the scope of invention disclosed herein.

1. A solid, ingestible composition comprising:
an alginate;
a bicarbonate; and
a carbonate
wherein the composition is in the form of a chewable core.

2. A composition as claimed in claim 1 wherein the chewable core is provided with a polysaccharide material.

3. A composition as claimed in claim 2 wherein the polysaccharide material is selected from the group consisting of xanthan gum, carrageenans, gum arabic, and cellulose derivatives.

4. A composition as in claim 3 wherein the polysaccharide material is xanthan gum.

5. A composition as claimed in claim 1 wherein said composition further comprises a coating.

6. A composition as claimed in claim 5 wherein the coating is selected from the group consisting of a hard coating, a soft coating and a shell.

7. A composition as claimed in claim 6 wherein the coating comprises xylitol.

8. A composition as claimed in claim 1 wherein said composition has a content of alginate of from 5 to 30 wt % based on the total weight of the composition.

9. A composition as claimed in claim 1 wherein said composition has a content of bicarbonate of 2 to 20 wt %.

10. A composition as claimed in claim 1 wherein said composition has a content of carbonate of 0.5 to 20 wt %.

11. A composition as claimed in claim 1 wherein the alginate is sodium alginate.

12. A composition as claimed in claim 1 wherein the bicarbonate is sodium bicarbonate.

13. A composition as claimed in claim 1 wherein the carbonate is calcium carbonate.

14. A composition as claimed in claim 1 wherein said composition is used in a method of treatment of the human or animal body by therapy for the treatment of one or more of reflux oesophagitis, gastritis, dyspepsia or peptic ulceration or for use as a sustained releasing or targeted delivery composition.

15. A composition as claimed in claim 14 wherein the composition is administered in an amount of from 100 to 2000 mg alginate per dose.

16. Use of a composition according to claim 1 in the manufacture of a medicament for the treatment of one or more of reflux oesophagitis, gastritis, dyspepsia or peptic ulceration or extra-oesophogeal gastric reflex conditions or for use as a sustained releasing or targeted delivery composition.

17. Use of a composition according to claim 1 for the treatment of one or more of reflux oesophagitis, gastritis, dyspepsia, peptic ulceration or extra-oesophageal gastric reflux conditions or for use as a sustained releasing or targeted delivery composition.

18. A process for preparing the composition as claimed in claim 1 comprising:
providing a mixture of excipients suitable for forming a core having a chewable texture;
heating the resulting mixture to a temperature of not greater than 50° C.;
adding an alginate, and blending the resulting mixture;
adding the bicarbonate and the carbonate, and blending the mixture;
forming the resulting mixture into the desired core product; and
coating the core with a suitable coating material.

19. A process as claimed in claim 18 wherein the temperature is selected to be not greater than 45° C.

20. A process as claimed in claim 18 wherein the water content of the mixture is selected such that the composition has the desired consistency.

21. A process as claimed in claim 18 wherein the process further comprises a first coating step wherein the core is pre-coated with a material.

22. A process as claimed in claim 21 wherein the material used to pre-coat the core is selected from the group consisting of xanthan gum, carrageenans, gum arabic, and cellulose derivatives.

23. A composition as claimed in claim 3 wherein the cellulose derivatives are selected from the group consisting of sodium carboxymethyl cellulose, pectins, tragacanth, and guar gum.

24. A process as claimed in claim 22 wherein the cellulose derivatives are selected from the group consisting of sodium carboxymethyl cellulose, pectins, tragacanth, and guar gum.
